(19) United States
(12) Patent Application Publication
(10) Pub. No.: US 2016/0194281 A1
(43) Pub. Date: Jul. 7, 2016

(54) CRYSTALLINE FORMS OF SILODOSIN

(71) Applicant: Johnson Matthey Public Limited Company, London (GB)

(72) Inventors: Aniruddh SINGH, Carteret, NJ (US); Mahmoud MIRMEHRABI, Ayer, MA (US)

(73) Assignee: Johnson Matthey Public Limited Company, London (GB)

(21) Appl. No.: 14/989,330

(22) Filed: Jan. 6, 2016

Related U.S. Application Data
(60) Provisional application No. 62/100,180, filed on Jan. 6, 2015.

(51) Int. Cl. C07D 209/12

(52) U.S. Cl. CPC C07D 209/12

(57) ABSTRACT

The present disclosure is directed to novel crystalline forms of silodosin and compositions comprising any of the novel crystalline forms of silodosin. Also provided are processes for the preparation of novel crystalline forms of silodosin.

### CROSS-REFERENCE

This application claims priority benefit to U.S. Provisional Patent Application No. 62/100,180 filed on Jan. 6, 2015, which is incorporated herein by reference.

### FIELD OF THE INVENTION

The present disclosure is directed to crystalline forms of silodosin. The present disclosure also describes processes for preparing the crystalline forms described herein.

### BACKGROUND OF THE INVENTION

2,3-Dihydro-1-(3-hydroxypropyl)-5-[(2R)-2-[[2-[2-(2,2,2-trifluoroethoxy)phenoxy]ethyl]amino] propyl]-1H-indole-7-carboxamide, hereinafter referred to as silodosin, acts as an al-adrenoceptor antagonist and is useful as a therapeutic agent for dysuria (EP 2474529). Silodosin is an indoline antidysuric which has a selective inhibitory effect against urethra smooth muscle constriction, and decreases urethra internal pressure without great influence on blood pressure (WO 2013/072935).

Silodosin is approved in the United States for 4 mg twice daily dosing and 8 mg once daily dosing to treat symptoms associated with benign prostatic hyperplasia (“BPH”) and is marketed under the brand name Rapaflo®. It is also marketed in Japan under the brand name Urief, in Europe under the brand name Silodyx and in India under the brand name Rapilif (EP 2474529).

Silodosin and its pharmaceutically acceptable salts are described in U.S. Pat. No. 5,387,603. Some crystalline forms of silodosin are known. EP 1541554 describes crystalline forms α, β and γ of silodosin as well as the preparation thereof. EP 2474529 describes crystalline forms δ and ε of silodosin and methods for the preparation thereof. EP 1541554 and EP 2474529 are incorporated herein by reference.

### SUMMARY OF THE INVENTION

The present disclosure is directed to three novel crystalline forms of silodosin and to processes for their preparation. These forms are identified herein as Forms A, B, and C. The present disclosure is further directed to pharmaceutical compositions comprising the crystalline forms of silodosin described herein.

### BRIEF DESCRIPTION OF THE DRAWINGS

FIG. 1 is an X-ray powder diffractogram of silodosin Form A, expressed in terms of ° 2θ.

FIG. 2 is an X-ray powder diffractogram of silodosin crystalline Form B, expressed in terms of ° 2θ.

FIG. 3 is an X-ray powder diffractogram of silodosin crystalline Form C, expressed in terms of ° 2θ.

FIG. 4 is a measured differential scanning calorimetry thermogram for silodosin Form A.

FIG. 5 is a measured differential scanning calorimetry thermogram for silodosin Form B.

FIG. 6 is a measured differential scanning calorimetry thermogram for silodosin Form C.

FIG. 7 is a thermal gravimetric analysis thermogram for silodosin Form A.

FIG. 8 is a thermal gravimetric analysis thermogram for silodosin Form B.

FIG. 9 is a thermal gravimetric analysis thermogram for silodosin Form C.

### DETAILED DESCRIPTION OF THE INVENTION

The present disclosure is directed to three novel crystalline forms of silodosin, as herein described in detail. More particularly, the present disclosure is directed to novel crystalline Forms A, B and C of silodosin.

The novel crystalline forms of silodosin of the present disclosure may be prepared directly or indirectly from silodosin Form β as described in EP 1541554 and/or may be interconverted from other crystalline forms of silodosin, including those described in EP 1541554 and EP 2474529. Examples 1-10 herein provide embodiments of the preparation of the crystalline forms of silodosin described in the present disclosure.

The novel crystalline forms of silodosin described herein may be characterized by one or more of their characteristic physical properties including, but not limited to, X-ray powder diffraction peaks, differential scanning calorimetry, and thermal gravimetric analyses.

X-ray powder diffraction analysis on representative samples of the crystalline forms of silodosin as herein described is performed using a Bruker D8 Advance instrument equipped with a Cu Kα radiation source (1.54° Angstrom), a 9-position sample holder and a LYNXEYE™ Super Speed Detector. Samples are placed on zero-background, silicon plate holders.

One skilled in the art would recognize that the ° 2θ values and the relative intensity values are generated by performing a peak search on the measured data and the d-spacing values are calculated by the instrument from the ° 2θ values using Bragg's equation. One skilled in the art would further recognize that the relative intensity for the measured peaks may vary as a result of sample preparation, orientation and instrument used, for example. A variation of about ±0.2 is not atypical in obtainable 2θ values.

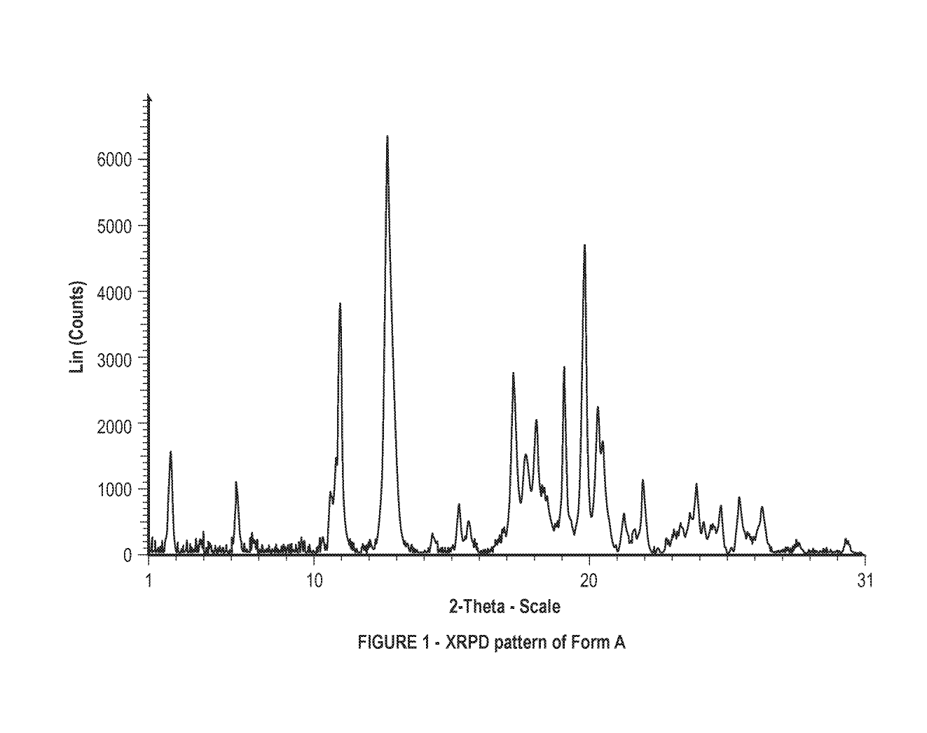

Silodosin Form A is a unique crystalline phase. Silodosin Form A is characterized by its X-ray powder diffraction pattern peaks and/or d-spacing values, as listed in Table 1 below. FIG. 1 is a representative X-ray powder diffractogram for a representative sample of silodosin Form A made according to Examples 1 and 6.

TABLE 1
XRPD peak list of Form A

| Angle, 2θ | d spacing | Intensity, % |
|---|---|---|
| 4.76 | 18.56 | 24.5 |
| 5.90 | 14.98 | 3.5 |
| 7.18 | 12.29 | 14.9 |
| 7.80 | 11.32 | 3.1 |
| 9.53 | 9.27 | 3.8 |
| 10.28 | 8.60 | 3.9 |
| 10.61 | 8.33 | 14.1 |
| 10.94 | 8.08 | 60 |
| 12.01 | 7.36 | 3.2 |
| 12.66 | 6.99 | 100 |
| 14.30 | 6.19 | 4.6 |
| 15.27 | 5.80 | 11.9 |
| 15.61 | 5.67 | 7.7 |
| 17.25 | 5.14 | 43.3 |
| 17.69 | 5.01 | 23.7 |
| 18.06 | 4.91 | 32.4 |
| 18.34 | 4.83 | 15.6 |
| 19.08 | 4.65 | 44.8 |
| 19.82 | 4.48 | 74 |
| 20.29 | 4.37 | 35.8 |
| 20.47 | 4.34 | 26.1 |
| 21.25 | 4.18 | 9.6 |
| 21.61 | 4.11 | 5.7 |
| 21.95 | 4.05 | 17.6 |
| 22.81 | 3.90 | 3.6 |
| 23.07 | 3.85 | 6.5 |
| 23.32 | 3.81 | 7.5 |
| 23.89 | 3.72 | 16.9 |
| 24.16 | 3.68 | 8.9 |
| 24.76 | 3.59 | 11.4 |
| 25.45 | 3.50 | 12.9 |
| 26.27 | 3.39 | 11.2 |
| 27.56 | 3.23 | 2.5 |
| 29.34 | 3.04 | 3 |

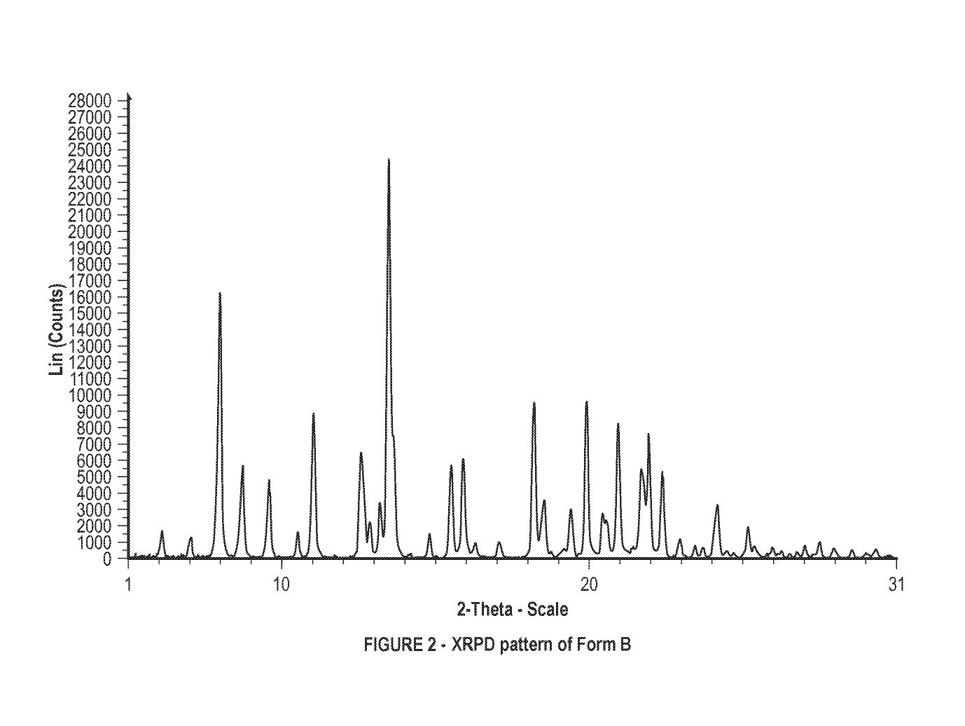

Silodosin crystalline Form B is a unique crystalline phase. Silodosin Form B is characterized by its X-ray powder diffraction pattern peaks and/or d-spacing values, as listed in Table 2 below. FIG. 2 is a representative X-ray powder diffractogram for a representative sample of silodosin Form B made according to Examples 2, 3, 4, 5 and 7.

TABLE 2
XRPD peak list of Form B

| Angle, 2θ | d spacing | Intensity, % |
|---|---|---|
| 5.11 | 17.27 | 6.5 |
| 6.07 | 14.56 | 5 |
| 7.06 | 12.51 | 63.6 |
| 7.83 | 11.29 | 22 |
| 8.72 | 10.13 | 18.6 |
| 9.70 | 9.11 | 6.2 |
| 10.23 | 8.64 | 34.5 |
| 11.84 | 7.47 | 25.5 |
| 12.15 | 7.28 | 9.2 |
| 12.47 | 7.09 | 13.3 |
| 12.78 | 6.92 | 100 |
| 14.14 | 6.26 | 5.9 |
| 14.87 | 5.95 | 22 |
| 15.28 | 5.79 | 23.8 |
| 15.69 | 5.65 | 3.6 |
| 16.49 | 5.37 | 3.9 |
| 17.68 | 5.01 | 37.4 |
| 18.00 | 4.92 | 14 |
| 18.90 | 4.69 | 11.7 |
| 19.43 | 4.57 | 37.2 |
| 20.02 | 4.43 | 10.1 |
| 20.50 | 4.33 | 32.2 |
| 21.29 | 4.17 | 21.3 |
| 21.54 | 4.12 | 30 |
| 21.99 | 4.04 | 20.6 |
| 22.57 | 3.94 | 4.5 |
| 23.09 | 3.85 | 3 |
| 23.34 | 3.81 | 2.7 |
| 23.82 | 3.73 | 12.8 |
| 24.87 | 3.58 | 7.4 |
| 25.07 | 3.55 | 2.5 |
| 25.68 | 3.47 | 2.7 |
| 26.80 | 3.32 | 3 |
| 27.29 | 3.27 | 3.9 |
| 27.77 | 3.21 | 2.4 |
| 28.39 | 3.14 | 2 |
| 29.17 | 3.06 | 2 |

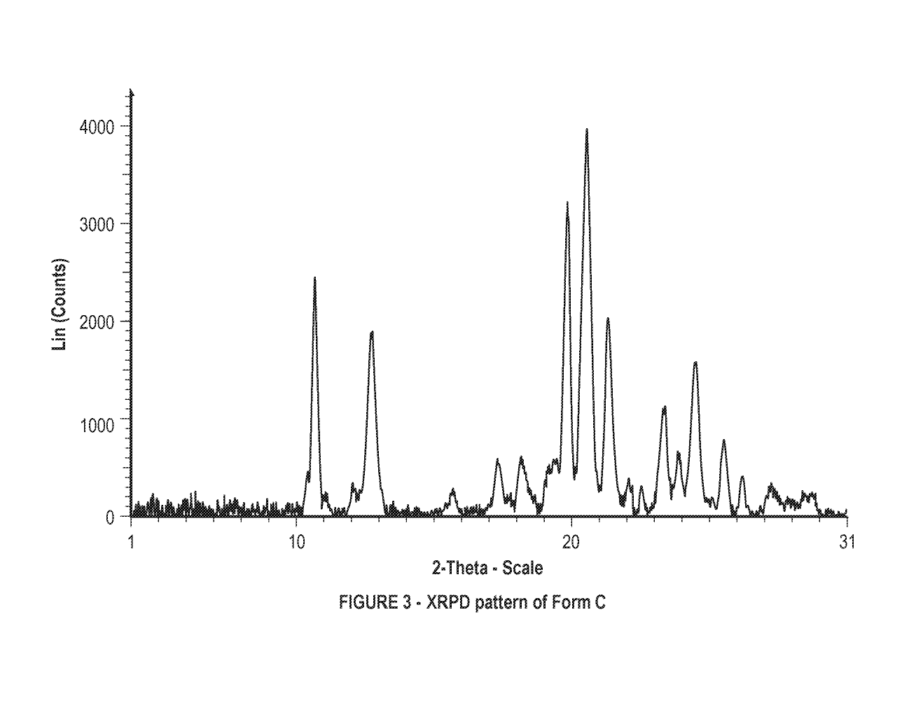

Silodosin crystalline Form C is a unique crystalline phase. Silodosin Form C is characterized by its X-ray powder diffraction pattern peaks and/or d-spacing values, as listed in Table 3 below. FIG. 3 is a representative X-ray powder diffractogram for a representative sample of silodosin Form C made according to Examples 8, 9 and 10.

TABLE 3
XRPD peak list of Form C

| Angle, 2θ | d spacing | Intensity, % |
|---|---|---|
| 10.67 | 8.29 | 61.5 |
| 12.07 | 7.33 | 7.9 |
| 12.73 | 6.95 | 47.7 |
| 15.68 | 5.65 | 7.2 |
| 17.33 | 5.11 | 14.4 |
| 18.17 | 4.88 | 15.4 |
| 19.17 | 4.63 | 12.9 |
| 19.44 | 4.56 | 14.4 |
| 19.85 | 4.47 | 80.8 |
| 20.56 | 4.32 | 100 |
| 21.34 | 4.16 | 51 |
| 22.08 | 4.02 | 9.5 |
| 22.55 | 3.94 | 7.7 |
| 23.36 | 3.81 | 28.3 |
| 23.90 | 3.72 | 16.5 |
| 24.50 | 3.63 | 39.6 |
| 25.54 | 3.48 | 19.6 |
| 26.22 | 3.40 | 10.2 |
| 27.27 | 3.27 | 8.4 |
| 28.47 | 3.13 | 6.2 |

Differential scanning calorimetry is performed using a TA Instruments Q10 DSC. Typically, samples are placed in unsealed, covered hermetic alodined aluminum sample pans and scanned from about 30° C. to about 300° C. at a rate of about 10° C./min under a nitrogen purge of about 50 mL/min.

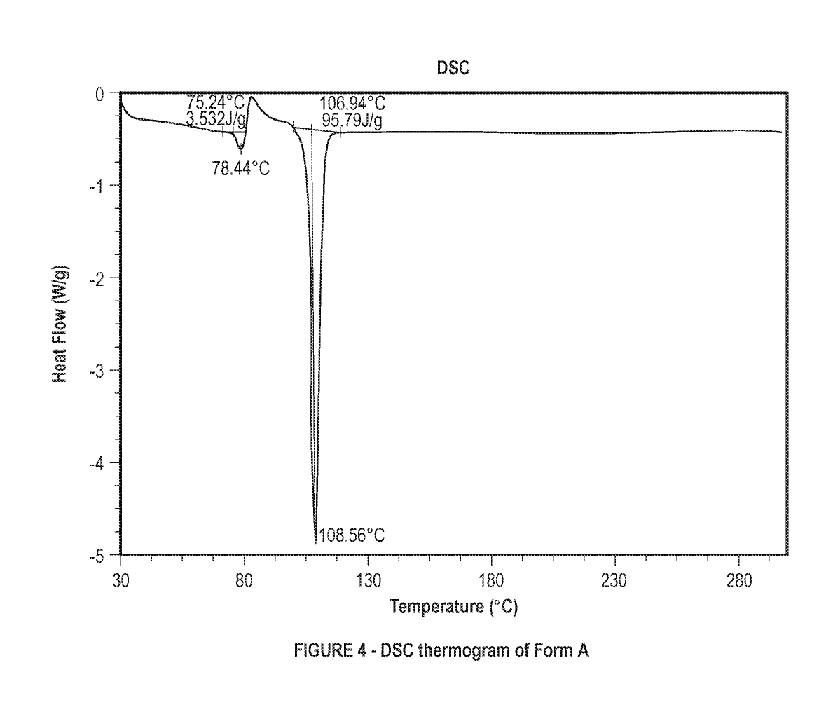

Differential scanning calorimetry is performed on representative samples of silodosin Forms A, B and C, as shown in FIGS. 4, 5 and 6, respectively. Two thermal events may be observed in the differential scanning calorimetry thermogram for silodosin Form A at about 78° C. and about 109° C. One thermal event at a peak of about 108° C. may be observed in the differential scanning calorimetry thermogram for silodosin Form B. Two thermal events may be observed in the differential scanning calorimetry thermogram for Form C at about 76° C. and about 107° C.

Thermal gravimetric analyses are performed using a TA Instruments TGA Q500. Typically samples are placed in an open, pre-tared aluminum sample pan and scanned from about 30° C. to about 300° C. at a rate of about 10° C./min using a nitrogen purge at about 60 mL/min.

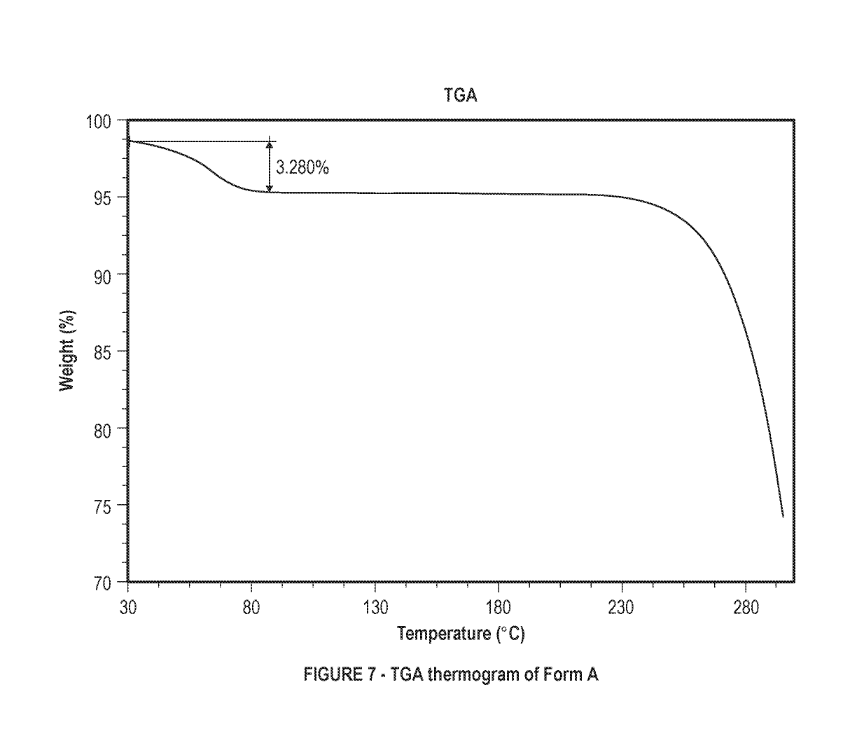

Thermal gravimetric analysis is performed on representative samples of silodosin Form A, B, and C, as shown in FIGS. 7, 8, and 9, respectively. Thermal gravimetric analysis data of Form A shows a weight loss of about 3.3% between about room temperature and about 80° C. Thermal gravimetric analysis data of Form B shows a weight loss of about 2.2% up to about 230° C. Thermal gravimetric analysis data of Form C shows a weight loss of about 0.7% up to about 230° C.

Any of the silodosin Forms A, B and/or C disclosed herein can be incorporated into various pharmaceutical dosage forms. Pharmaceutical compositions may comprise any of the silodosin Forms A, B and/or C and a pharmaceutically acceptable carrier. The pharmaceutical compositions may further comprise one or more pharmaceutically acceptable excipients. Suitable excipients may be selected from the group consisting of fillers, sweeteners, buffering agents, glidants, flowing agents, flavouring agents, lubricants, preservatives, surfactants, wetting agents, binders, disintegrants and thickeners. However, other excipients may also be used. Examples of suitable excipients are described, for example, in EP 1574215, EP 2474529, WO 2013061338 and WO 2014006635. Rapaflo® capsules for oral administration contain silodosin, and the following inactive ingredients: D-mannitol, magnesium stearate, pregelatinized starch, and sodium lauryl sulfate. Rapaflo® hard gelatin capsules contain gelatin and titanium dioxide. Any of the silodosin Forms A, B and/or C can be formulated as tablet, capsule, or any formulation known to those skilled in the art. Examples of suitable processes for the preparation of pharmaceutical compositions are described, for example, in EP 1574215, EP 2474529, WO 2013061338 and WO 2014006635. These examples can be repeated using any of the silodosin Forms A, B and/or C disclosed herein.

In a general aspect, the present disclosure provides for a method of treating dysuria, benign prostatic hyperplasia and related diseases by administering to a human patient a pharmaceutical composition comprising one or more of the forms of silodosin described herein.

### EXAMPLES

The invention is illustrated by the following examples.

The following examples are set forth to aid in the understanding of the invention, and are not intended and should not be construed to limit in any way the invention set forth in the claims which follow thereafter.

### Example 1

### Preparation of Form A Using Methyl Ethyl Ketone

About 75 mg of silodosin Form β and about 0.75 mL of methyl ethyl ketone are added to a vial. The mixture is slurried overnight at about 20 to 25° C. The slurry is centrifuged. The resulting solids are analyzed by X-ray powder diffraction and determined to be silodosin Form A.

### Example 2

### Preparation of Form B Using Isopropyl Acetate

About 75 mg of silodosin Form β and about 0.75 mL of isopropyl acetate are added to a vial. The mixture is slurried overnight at about 20 to 25° C. The slurry is centrifuged. The resulting solids are analyzed by X-ray powder diffraction and determined to be silodosin Form B.

### Example 3

### Preparation of Form B Using Isopropyl Acetate

About 75 mg of silodosin Form β and about 0.75 mL of isopropyl acetate are added to a vial. The mixture is slurried overnight at about 40 to 50° C. The slurry is centrifuged. The resulting solids are analyzed by X-ray powder diffraction and determined to be silodosin Form B.

### Example 4

### Preparation of Form B Using Cyclopentyl Methyl Ether

About 75 mg of silodosin Form β and about 0.75 mL of cyclopentyl methyl ether are added to a vial. The mixture is slurried overnight at about 40 to 50° C. The slurry is centrifuged. The resulting solids are analyzed by X-ray powder diffraction and determined to be silodosin Form B.

### Example 5

### Preparation of Form B Using Methyltetrahydrofuran

About 75 mg of silodosin Form β and about 0.75 mL of methyltetrahydrofuran are added to a vial. The mixture is slurried overnight at about 40 to 50° C. The slurry is centrifuged. The resulting solids are analyzed by X-ray powder diffraction and determined to be silodosin Form B.

### Example 6

### Preparation of Form A Using 3-Pentanone

About 75 mg of silodosin Form β and about 0.75 mL of 3-pentanone are added to a vial. The mixture is slurried overnight at about 20 to 25° C. The slurry is centrifuged. The resulting supernatant is added to a vial. The supernatant is evaporated to dryness under vacuum at about 20 to 25° C. The resulting solids are analyzed by X-ray powder diffraction and determined to be silodosin Form A.

### Example 7

### Preparation of Form B Using Cyclopentyl Methyl Ether

About 75 mg of silodosin Form β and about 0.75 mL of cyclopentyl methyl ether are added to a vial. The mixture is heated on a stir plate to about 60° C. to dissolve the silodosin. The stir plate is turned off and the solution is cooled naturally to about 20 to 25° C. The resulting slurry is filtered. The resulting solids are analyzed by X-ray powder diffraction and determined to be silodosin Form B.

### Example 8

### Preparation of Form C Using Toluene

About 75 mg of silodosin Form β and about 0.75 mL of toluene are added to a vial. The mixture is heated on a stir plate to about 60° C. to dissolve the silodosin. The solution is removed from the stir plate and immediately transferred to an ice bath. The resulting slurry is filtered. The resulting solids are analyzed by X-ray powder diffraction and determined to be silodosin Form C.

### Example 9

### Preparation of Form C Using 3-Pentanone

About 75 mg of silodosin Form β and about 0.75 mL of 3-pentanone are added to a vial. The mixture is heated on a stir plate to about 60° C. to dissolve the silodosin. The solution is removed from the stir plate and immediately transferred to an ice bath. The resulting slurry is filtered. The resulting solids are analyzed by X-ray powder diffraction and determined to be silodosin Form C.

### Example 10

### Preparation of Form C Using Methyl Ethyl Ketone

About 75 mg of silodosin Form β and about 0.75 mL of methyl ethyl ketone are added to a vial. The mixture is heated on a stir plate to about 60° C. to dissolve the silodosin. The solution is removed from the stir plate and immediately transferred to an ice bath. The resulting slurry is filtered. The resulting solids are analyzed by X-ray powder diffraction and determined to be silodosin Form C.

What is claimed is:

1. A form of silodosin selected from the group consisting of Form A, Form B and Form C; wherein:
Form A has an X-ray powder diffraction pattern comprising peaks, in terms of 2-theta, at about 12.66 and about 19.82;
Form B has an X-ray powder diffraction pattern comprising peaks, in terms of 2-theta, at about 7.06 and about 12.78; and
Form C has an X-ray powder diffraction pattern comprising peaks, in terms of 2-theta, at about 19.85 and about 20.56.

2. A form of silodosin according to claim 1, wherein:
Form A has an X-ray powder diffraction pattern further comprising peaks, in terms of 2-theta, at about 10.94, about 17.25 and about 19.08;
Form B has an X-ray powder diffraction pattern further comprising peaks, in terms of 2-theta, at about 10.23, about 17.68 and about 19.43; and
Form C has an X-ray powder diffraction pattern further comprising a peak, in terms of 2-theta, at about 10.67, about 12.73 and about 21.34.

3. A form of silodosin selected from the group consisting of Form A, Form B and Form C; wherein:
Form A has an X-ray powder diffraction pattern substantially as shown in FIG. 1;
Form B has an X-ray powder diffraction pattern substantially as shown in FIG. 2; and
Form C has an X-ray powder diffraction pattern substantially as shown in FIG. 3.

4. A form of silodosin selected from the group consisting of Form A, Form B and Form C; wherein:
Form A has an X-ray powder diffraction pattern comprising peaks at d-spacing, in terms of Angstroms, of about 6.99 and about 4.48;
Form B has an X-ray powder diffraction pattern comprising peaks at d-spacing, in terms of Angstroms, of about 12.51 and about 6.92; and
Form C has an X-ray powder diffraction pattern comprising peaks at d-spacing, in terms of Angstroms, of about 4.47 and about 4.32.

5. A form of silodosin according to claim 4, wherein:
Form A has an X-ray powder diffraction pattern further comprising peaks at d-spacing, in terms of Angstroms, of about 8.08, about 5.14 and about 4.65;
Form B has an X-ray powder diffraction pattern further comprising peaks at d-spacing, in terms of Angstroms, of about 8.64, about 5.01 and about 4.57; and
Form C has an X-ray powder diffraction pattern comprising further a peak at d-spacing, in terms of Angstroms, of about 8.29, about 6.95 and about 4.16.

6. A form of silodosin according to claim 1, wherein, as measured by differential scanning calorimetry:
Form A is characterized by an endothermic event at about 109° C.;
Form B is characterized by an endothermic event at about 108° C.; and
Form C is characterized by an endothermic event at about 107° C.

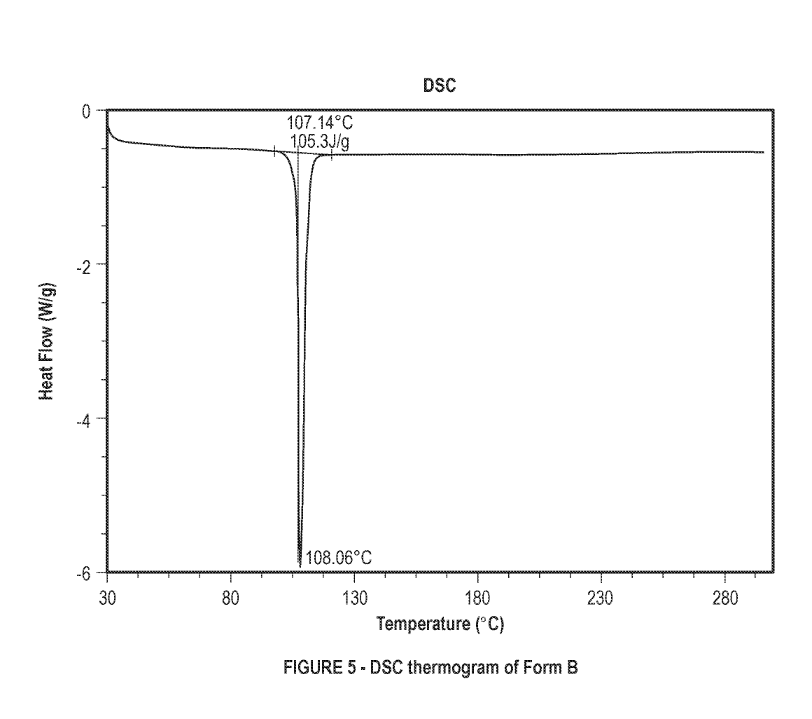

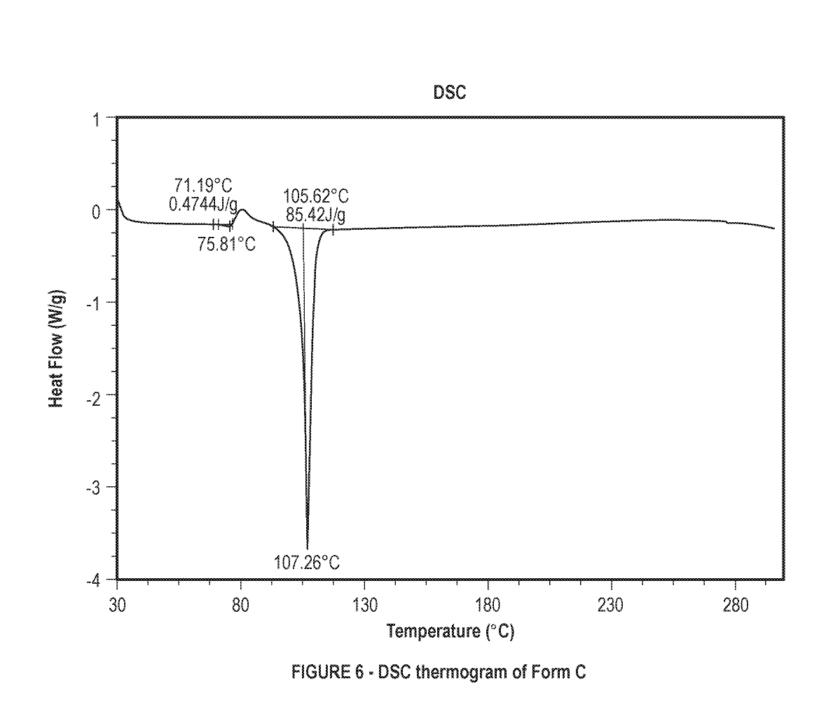

7. A form of silodosin according to claim 1, wherein:
Form A is characterized by a differential scanning calorimetry pattern substantially as shown in FIG. 4;
Form B is characterized by a differential scanning calorimetry pattern substantially as shown in FIG. 5; and
Form C is characterized by a differential scanning calorimetry pattern substantially as shown in FIG. 6.

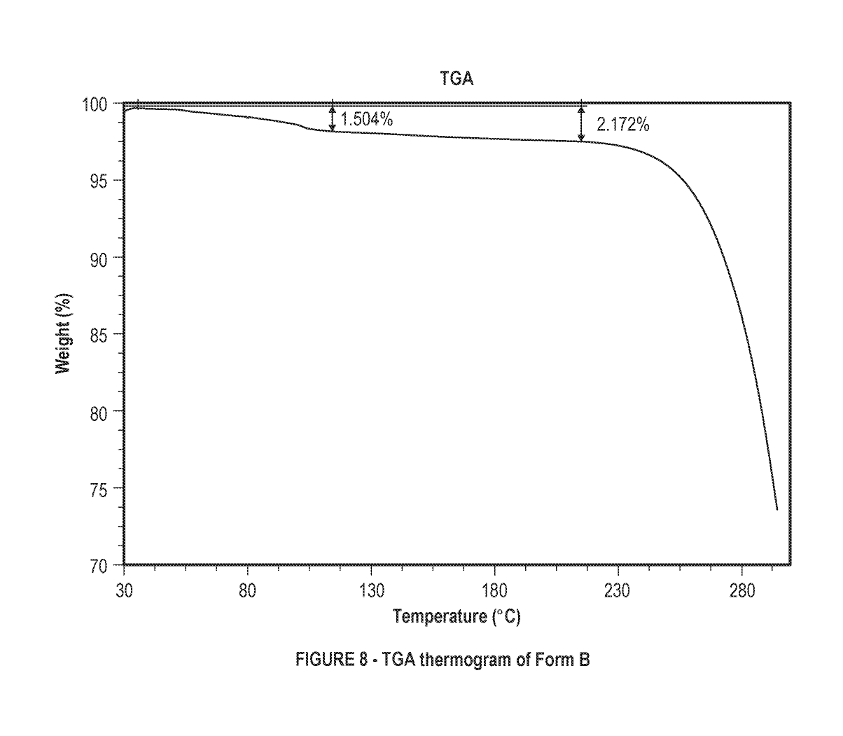

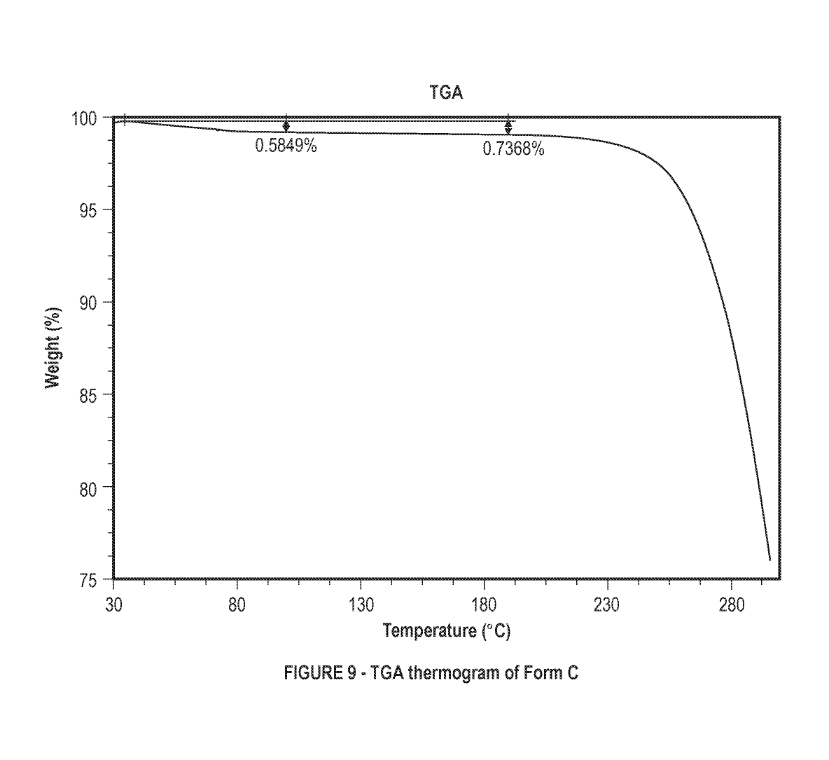

8. A form of silodosin according to claim 1, wherein:
Form A is characterized by a thermal gravimetric analysis pattern substantially as shown in FIG. 7;
Form B is characterized by a thermal gravimetric analysis pattern substantially as shown in FIG. 8; and
Form C is characterized by a thermal gravimetric analysis pattern substantially as shown in FIG. 9.

9. A pharmaceutical formulation comprising at least one pharmaceutically acceptable excipient and silodosin according to claim 1.

10. The pharmaceutical formulation according to claim 9, wherein the formulation is an oral dosage form.

11. A method of treating dysuria comprising administering a pharmaceutical formulation according to claim 9 to a patient in need thereof.

12. A method of treating benign prostatic hyperplasia comprising administering a pharmaceutical formulation according to claim 9 to a patient in need thereof.

13. A method of making silodosin Form A according to claim 1, comprising exposing a starting material comprising silodosin Form β to methyl ethyl ketone at a temperature in the range of about 20 to 25° C. for a time sufficient to yield silodosin Form A.

14. A method of making silodosin Form B according to claim 1, comprising exposing a starting material comprising silodosin Form β to isopropyl acetate at a temperature in the range of about 20 to 25° C. for a time sufficient to yield silodosin Form B.

15. A method of making silodosin Form B according to claim 1, comprising exposing a starting material comprising silodosin Form β to isopropyl acetate at a temperature in the range of about 40 to 50° C. for a time sufficient to yield silodosin Form B.

16. A method of making silodosin Form B according to claim 1, comprising exposing a starting material comprising silodosin Form β to cyclopentyl methyl ether at a temperature in the range of about 40 to 50° C. for a time sufficient to yield silodosin Form B.

17. A method of making silodosin Form B according to claim 1, comprising exposing a starting material comprising silodosin Form β to methyltetrahydrofuran at a temperature in the range of about 40 to 50° C. for a time sufficient to yield silodosin Form B.

18. A method of making silodosin Form A according to claim 1, comprising exposing a starting material comprising silodosin Form β to 3-pentanone at a temperature in the range of about 20 to 25° C. and evaporating to dryness to yield silodosin Form A.

19. A method of making silodosin Form B according to claim 1, comprising exposing a starting material comprising silodosin Form β to cyclopentyl methyl ether at a temperature in the range of about 55 to 65° C. followed by cooling to a temperature in the range of about 20 to 25° C. to yield silodosin Form B.

20. A method of making silodosin Form C according to claim 1, comprising exposing a starting material comprising silodosin Form β to toluene at a temperature in the range of about 55 to 65° C. followed by immediate cooling to a temperature in the range of about 20 to 25° C. to yield silodosin Form C.

21. A method of making silodosin Form C according to claim 1, comprising exposing a starting material comprising silodosin Form β to 3-pentanone at a temperature in the range of about 55 to 65° C. followed by immediate cooling to a temperature in the range of about 20 to 25° C. to yield silodosin Form C.

22. A method of making silodosin Form C according to claim 1, comprising exposing a starting material comprising silodosin Form β to methyl ethyl ketone at a temperature in the range of about 55 to 65° C. followed by immediate cooling to a temperature in the range of about 20 to 25° C. to yield silodosin Form C.
